(19) United States
(12) Patent Application Publication
(10) Pub. No.: US 2015/0057357 A1
(43) Pub. Date: Feb. 26, 2015

(54) BIS (THIOHYDRAZIDE AMIDE) COMPOUNDS FOR TREATING CANCERS

(71) Applicant: The Board of Trustees of The Leland Stanford Junior University, Palo Alto, CA (US)

(72) Inventors: Elizabeth Alli, Cupertino, CA (US); James Ford, Mountain View, CA (US)

(21) Appl. No.: 14/370,631

(22) PCT Filed: Jan. 4, 2013
(86) PCT No.: PCT/US2013/020257

§ 371 Date: Jul. 3, 2014

Related U.S. Application Data
(60) Provisional application No. 61/583,396, filed on Jan. 5, 2012.

(51) Int. Cl. A61K 31/166; A61K 45/06

(52) U.S. Cl. CPC A61K 31/166; A61K 45/06
USPC 514/599

(57) ABSTRACT

Methods of treating a subject with cancer characterized by an impaired ability to repair oxidative DNA damage, comprising administering to the subject an effective amount of a bis(thiohydrazide amide) compound are provided.

### RELATED APPLICATIONS

This application claims priority to U.S. Provisional Application No. 61/583,396, filed on Jan. 5, 2012, the entire contents of which are hereby incorporated herein by reference.

### BACKGROUND OF THE INVENTION

Many cancers are associated with aggressive phenotypes and high rates of relapse following chemotherapy. Among such cancers are certain subtypes of breast cancer, for example, basal-like breast cancer. The basal-like breast cancer subtype can sometimes include hereditary breast cancers due to germline mutations in the Breast Cancer Susceptibility Gene 1 (BRCA1). Basal-like sporadic breast cancers and BRCA1-mutated hereditary breast cancers frequently assume triple-negative status, i.e. lack the expression of estrogen receptor-α (ER-α) and progesterone receptor (PR) and lack the amplification/overexpression of the HER2/Neu oncogene. It was also recently demonstrated that basal-like breast cancer subtypes are characterized by the impaired ability to repair oxidative DNA damage (Alli et al., Defective repair of oxidative DNA damage in triple-negative breast cancer confers sensitivity to inhibition of Poly(ADP-ribose) polymerase, Cancer Res., 2009, 69, 3589-96.) Because basal-like breast cancers are associated with aggressive phenotypes, and triple-negative breast cancers are also insensitive to hormonal manipulation and Her2-targeting therapies, improved methods for treating aggressive cancers are needed.

### SUMMARY OF THE INVENTION

It was surprisingly found that cancers with aggressive phenotype, such as basal-like breast cancers, are selectively sensitive to treatment with bis(thiohydrazide amide) compounds. Specifically, it was demonstrated that a bis(thiohydrazide amide compound), elesclomol, when used at concentrations of up to 1 μM, induced cell death in up to 75% of basal-like breast cancer cells and in up to 95% of BRCA1-mutated breast cancer cells, while normal breast cells and luminal breast cancer cells remained unaffected by elesclomol treatment (see Example 1, FIG. 2).

In one embodiment, the present invention is a method of treating a subject with cancer characterized by an impaired ability to repair oxidative DNA damage. The method comprises administering to the subject an effective amount of a bis(thiohydrazide amide) compound, or a pharmaceutically acceptable salt thereof, or a deprotonated form thereof complexed to a transition metal cation. The bis(thiohydrazide amide compound) is represented by the following Structural Formula:

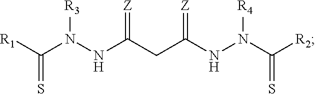

wherein:
R₁ and R₂ are each, independently, a phenyl optionally substituted with one or more groups selected from alkyl, haloalkyl, alkoxy, haloalkoxy, carbonyloxy, halogen, cyano, and nitro;
R₃ and R₄ are each, independently, —H or an alkyl;
each Z is independently O or S.

In some embodiments, R₃ and R₄ are both methyl and each Z is O. In further embodiments, R₁ and R₂ are each phenyl optionally substituted with one or more groups consisting of methyl, ethyl, methoxy, ethoxy, halogen, or hydroxyl. In some embodiments, R₁ and R₂ are each unsubstituted phenyl, and the bis(thiohydrazide amide is elesclomol represented by the following Structural Formula:

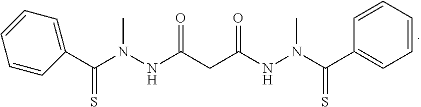 [SMILES: CN(NC(=O)CC(=O)NN(C)C(=S)c1ccccc1)C(=S)c1ccccc1]

In some embodiments, the cancer characterized by an impaired ability to repair oxidative DNA damage is breast cancer. In further embodiments, the breast cancer is BRCA1-mutated breast cancer, basal-like breast cancer or triple-negative breast cancer. In some embodiments, the impaired ability to repair oxidative DNA damage comprises impaired base excision repair (BER).

In some embodiments, the bis(thiohydrazide amide) compound is administered in combination with another chemotherapeutic agent. In further embodiments, the agent is selected from the group consisting of cisplatin, gemcitabine, paclitaxel, or a PARP inhibitor.

In some embodiments, the invention also provides a method of treating cancer in a subject in need thereof, comprising the steps of:
a) assessing the ability of the cancer to repair oxidative DNA damage;
b) if the cancer is sensitive to bis(thiohydrazide amide) compounds, treating the subject with cancer by administering bis(thiohydrazide amide) compounds; and
c) if the cancer is not sensitive to bis(thiohydrazide amide) compounds, treating the subject with cancer by administering an anti-cancer therapy that does not comprise bis(thiohydrazide amide) compounds.

In some embodiments, the cancer is breast cancer. In further embodiments, the breast cancer is BRCA1-mutated hereditary breast cancer, basal-like sporadic breast cancer, or triple-negative breast cancer.

### BRIEF DESCRIPTION OF THE DRAWINGS

FIG. 1 is a series of images of breast cancer cell cultures after their treatment with increasing concentrations of elesclomol.

FIG. 2 is a cellular viability dose-response curve for normal or cancerous breast cells treated with increasing concentrations of elesclomol.

FIG. 3 is a cellular viability dose response curve for cell lines deficient in base excision repair enzyme and their isogenic controls treated with increasing concentrations of elesclomol.

### DETAILED DESCRIPTION OF THE INVENTION

### The Compound and Formulations Thereof

The current invention is directed to methods of treating a subject with cancer comprising administering to the subject an effective amount of a bis(thiohydrazide amide) compound, or a pharmaceutically acceptable salt thereof, or a deprotonated form thereof complexed to a transition metal cation. In some embodiments, the cancer is characterized by the impaired ability to repair oxidative DNA damage. The bis(thiohydrazide amide compound to be administered to a subject with cancer is represented by the following Structural Formula:

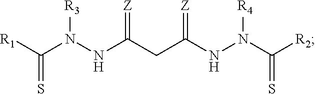

wherein:
R₁ and R₂ are each, independently, a phenyl optionally substituted with one or more groups selected from alkyl, haloalkyl, alkoxy, haloalkoxy, carbonyloxy, halogen, cyano, and nitro;
R₃ and R₄ are each, independently, —H or an alkyl;
each Z is independently O or S.

In some embodiments, R₃ and R₄ are both methyl, and each Z is O. In some embodiments, R₁ and R₂ are each phenyl optionally substituted with one or more groups consisting of methyl, ethyl, methoxy, ethoxy, halogen, or hydroxy. In one embodiment, R₃ and R₄ are both methyl, each Z is O, and R₁ and R₂ are each phenyl optionally substituted with one or more groups consisting of methyl, ethyl, methoxy, ethoxy, halogen, or hydroxy. In a further embodiment, R₁ and R₂ are each unsubstituted phenyl, and the bis(thiohydrazide amide) compound is elesclomol represented by the following structural formula:

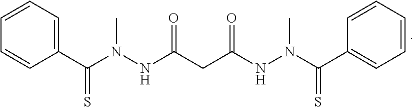 [SMILES: CN(NC(=O)CC(=O)NN(C)C(=S)c1ccccc1)C(=S)c1ccccc1]

The bis(thiohydrazide amide) compounds described herein may be present in the form of a pharmaceutically acceptable salt. Pharmaceutically acceptable salt forms include pharmaceutically acceptable basic/cationic salts. Basic addition salts include those derived from inorganic bases, such as ammonium or alkali or alkaline earth metal hydroxides, carbonates, bicarbonates, and the like, and organic bases such as alkoxides, alkyl amides, alkyl and aryl amines, and the like. Such bases useful in preparing the salts of this invention thus include sodium hydroxide, potassium hydroxide, ammonium hydroxide, potassium carbonate, and the like.

In some embodiments, a bis(thiohydrazide amide) compound is in a form of a disalt represented by the following structural formula:

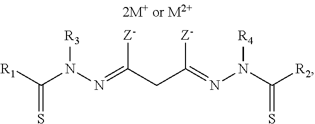

wherein R₁, R₂, R₃, R₄ and Z are as defined above.

M⁺ is a pharmaceutically acceptable monovalent cation and M²⁺ is a pharmaceutically acceptable divalent cation. “Pharmaceutically acceptable” means that the cation is suitable for administration to a subject. Examples of M⁺ or M²⁺ include Li⁺, Na⁺, K⁺, Mg²⁺, Ca²⁺, Zn²⁺, and NR₄⁺, wherein each R is independently hydrogen, a substituted or unsubstituted alkyl group (e.g., a hydroxyalkyl group, aminoalkyl group or ammoniumalkyl group). More preferably, the pharmaceutically acceptable cation is Na⁺ or K⁺. Na⁺ is even more preferred. In one embodiment, the bis(thiohydrazide amide) compound is a disalt represented by the following structural formula:

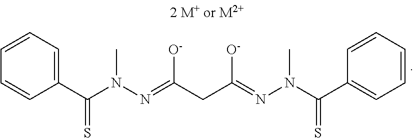 [SMILES: CN(/N=C(\[O-])C/C([O-])=N/N(C)C(=S)c1ccccc1)C(=S)c1ccccc1]

In some embodiments, a bis(thiohydrazide amide) compound can be in a deprotonated form complexed to transition metal ions. The term “complexed” means that the a bis(thiohydrazide amide) compound attaches to the a transition metal ion through one or more coordinate covalent bonds or coordination bonds. The term “chelated” means that the bis(thiohydrazide amide) compound binds to the transition metal ion at two or more attachment points through coordinate covalent bonds or coordination bonds. The terms “coordinate”, “coordinated”, “coordinate covalent bond” and “coordination bond” have the meanings that are commonly known to one of ordinary skill in the art. A “deprotonated form” of a bis(thiohydrazide amide) compound refers to a molecule wherein one or more protons from the bis(thiohydrazide amide) compound or a salt, hydrate, solvate or polymorph thereof have been removed. A deprotonated form of the bis(thiohydrazide amide) compound is represented by the following structural formula:

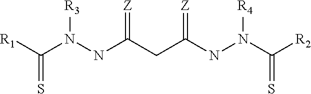

wherein R₁, R₂, R₃, R₄ and Z are as defined above.

A “transition metal cation” refers to a positively charged ion of a metal in Groups 3-12 of the Periodic Table. Examples include Ni²⁺, Cu⁺, Cu²⁺, Co²⁺, Co³⁺, Fe²⁺, Fe³⁺, Zn²⁺, Pt²⁺, Pd²⁺, V⁴⁺, V⁵⁺, Cr²⁺, Cr³⁺, Cr⁴⁺, Mn²⁺, Mn³⁺, Mn⁴⁺ and Mn⁵⁺. In a specific embodiment, the transition metal cations have a +2 charge. Examples include Ni²⁺, Cu²⁺, Co²⁺, Fe²⁺, Zn²⁺, Pt²⁺ and Pd²⁺. In a specific embodiment, the transition metal cation is Cu⁺, Cu²⁺ or Ni²⁺. In a more specific embodiment, the transition metal cation is Cu²⁺. The molar ratio of the bis(thiohydrazide amide) compound or a salt, hydrate, solvate, polymorph or a deprotonated form thereof to transition metal cation recited in this paragraph is, for example, equal to or greater than 0.5 and equal to or less than 2.0 (i.e. 0.5≦ratio≦2.0) or 1:1.

In one embodiment, a bis(thiohydrazide amide) compound complexed to a transition metal ion is represented by the following structural formula:

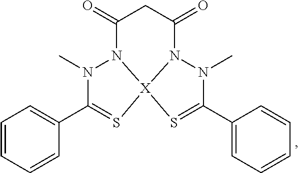

or an isomer, ester, salt thereof, wherein X is a transition metal cation having a +2 charge. In a preferred embodiment, X is Cu²⁺

The bis(thiohydrazide amide) compounds are preferably in a substantially pure form, e.g., greater than 50%, 60%, 70%, 80%, 90%, 95%, 97%, 99%, 99.5% or 99.9% pure by weight. “Percent purity by weight” means the weight of a bis(thiohydrazide amide) compound divided by the weight of the bis(thiohydrazide amide) compound plus impurities times 100%.

The bis(thiohydrazide amide) compounds described herein can be prepared according to methods described in U.S. Pat. Nos. 6,800,660, 6,762,204, and 6,825,235, and U.S. Publication No. 2008/0146842.

The disalts of bis(thiohydrazide amide) compounds can be prepared according to methods described in U.S. Pat. Nos. 7,385,084, 7,579,503, 7,795,313, and 8,048,925.

The transition metal complex of bis(thiohydrazide amide) compounds can be prepared according to methods described in WO 2010/048284 and WO 2010/048293, the entire contents of which are incorporated herein by reference.

The term “alkyl” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. The term “(C₁-C₆)alkyl” means a saturated straight chain or branched non-cyclic hydrocarbon having from 1 to 6 carbon atoms. Representative (C₁-C₆)alkyl groups are those shown above having from 1 to 6 carbon atoms. Alkyl groups included in compounds of this invention may be optionally substituted with one or more substituents.

The term “haloalkyl” means and alkyl group in which one or more (including all) the hydrogen radicals are replaced by a halo group, wherein each halo group is independently selected from —F, —Cl, —Br, and —I. The term “halomethyl” means a methyl in which one to three hydrogen radical(s) have been replaced by a halo group. Representative haloalkyl groups include trifluoromethyl, bromomethyl, 1,2-dichloroethyl, 4-iodobutyl, 2-fluoropentyl, and the like.

An “alkoxy” is an alkyl group which is attached to another moiety via an oxygen linker.

A “carbonyloxy” is a carbonyl group which is attached to another moiety via an oxygen linker.

A “haloalkoxy” is a haloalkyl group which is attached to another moiety via an oxygen linker.

An “alkylamino” is an amino group in which one of the hydrogens is replaced with an alkyl group.

A “dialkylamino” is an amino group in which two of the hydrogens are replaced with an alkyl groups. The alkyl groups may be the same or different.

The term “lower” refers to a group having up to four carbon atoms. For example, a “lower alkyl” refers to an alkyl radical having from 1 to 4 carbon atoms, “lower alkoxy” refers to “—O—(C₁-C₄)alkyl.

Suitable substituents for alkyl and alkoxy groups include lower alkyl, halo, cyano, nitro, amino, mono(lower alkyl)amino, di(lower alkyl)amino, lower alkoxy, lower haloalkoxy and hydroxy.

A bis(thiohydrazide amide) compound may be formulated as a pharmaceutical composition also comprising a pharmaceutically acceptable carrier or diluent. As used herein, a “pharmaceutical composition” can be a formulation containing the disclosed compounds, in a form suitable for administration to a subject. Suitable pharmaceutically acceptable carriers may contain inert ingredients which do not inhibit the biological activity of the bis(thiohydrazide amide) compounds. The pharmaceutically acceptable carriers should be biocompatible, i.e., non-toxic, non-inflammatory, non-immunogenic and devoid of other undesired reactions upon the administration to a subject. Standard pharmaceutical formulation techniques can be employed, such as those described in Remington: the Science and Practice of Pharmacy, 19^{th} edition, Mack Publishing Co., Easton, Pa. (1995). Exemplary pharmaceutical compositions comprising bis(thiohydrazide amide) compounds are described in U.S. Pat. No. 7,678,832.

The pharmaceutical composition can be in bulk or in unit dosage form. The unit dosage form can be in any of a variety of forms, including, for example, a capsule, an IV bag, a tablet, a single pump on an aerosol inhaler, or a vial. The quantity of a bis(thiohydrazide amide) compound in a unit dose is the effective amount of the bis(thiohydrazide amide) compound that can vary according to the chosen administration route. A variety of routes are contemplated, including topical, oral, transmucosal or parenteral, including transdermal, subcutaneous, intravenous, intramuscular, intraperitoneal and intranasal. For oral administration, a bis(thiohydrazide amide) compound can be combined with a suitable solid or liquid carrier or diluent to form capsules, tablets, pills, powders, syrups, solutions, suspensions, or the like.

The tablets, pills, capsules, and the like can contain from about 1 to about 99 weight percent of the active ingredient and a binder such as gum tragacanth, acacias, corn starch or gelatin; excipients such as dicalcium phosphate; a disintegrating agent such as corn starch, potato starch or alginic acid; a lubricant such as magnesium stearate; and/or a sweetening agent such as sucrose, lactose or saccharin. When a dosage unit form is a capsule, it may contain, in addition to materials of the above type, a liquid carrier such as a fatty oil.

For parental administration, a bis(thiohydrazide amide) compound can be combined with sterile aqueous or organic media to form injectable solutions or suspensions. For example, solutions in sesame or peanut oil, aqueous propylene glycol and the like can be used, as well as aqueous solutions of water-soluble pharmaceutically-acceptable salts of the compounds. Dispersions can also be prepared in glycerol, liquid polyethylene glycols and mixtures thereof in oils. Under ordinary conditions of storage and use, these preparations contain a preservative to prevent the growth of microorganisms.

In addition to the formulations described above, a formulation can optionally include, or be co-administered with one or more additional drugs, e.g., other antifungals, anti-inflammatories, antibiotics, antivirals, immunomodulators, antiprotozoals, steroids, decongestants, bronchodilators, antihistamines, anticancer agents, and the like. For example, the disclosed compounds can be co-administered with drugs such as such as ibuprofen, prednisone (corticosteroid) pentoxifylline, Amphotericin B, Fluconazole, Ketoconazol, Itraconazole, penicillin, ampicillin, amoxicillin, and the like. The formulation may also contain preserving agents, solubilizing agents, chemical buffers, surfactants, emulsifiers, colorants, odorants and sweeteners.

In general, the recommended daily dose range of bis(thiohydrazide amide) compound for the conditions described herein lie within the range of from about 0.01 mg to about 3000 mg per day. Specifically, a daily dose range should be from about 5 mg to about 500 mg per day, more specifically, between about 10 mg and about 200 mg per day. Suitable dosages for bis(thiohydrazide amide) compounds are also described in U.S. Pat. No. 8,017,654. In managing the patient, the therapy should be initiated at a lower dose, perhaps about 1 mg to about 25 mg, and increased if necessary up to about 200 mg to about 1000 mg per day as either a single dose or divided doses. It may be necessary to use dosages of bis(thiohydrazide amide) compound outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

### Methods of Treating Cancer by the Compounds of the Invention

It was surprisingly found that cancers with aggressive phenotype, such as basal-like breast cancers, that are also characterized by an impaired ability to repair oxidative DNA damage, are selectively killed by treatment with bis(thiohydrazide amide) compounds. Accordingly, the present invention is a method of treating cancer in a subject, the method comprising administering to the subject an effective amount of a bis(thiohydrazide amide) compound, wherein the cancer is characterized by an impaired ability to repair oxidative DNA damage.

The term “oxidative DNA damage” refers to all chemical modifications to DNA that can occur upon exposure of DNA to oxidizing agents. The oxidizing agents can be Reactive Oxygen Species (ROS) that are formed endogenously during normal cellular metabolic processes. Non-limiting examples of ROS include hydrogen peroxide (H₂O₂), superoxide (O₂^{•-}) and hydroxyl radical (^{•}OH). Oxidizing agents can react with DNA bases, e.g., adenine, cytosine, guanine or thymine, leading to eventual formation of DNA base lesions, e.g., chemically modified DNA bases. An example of DNA base lesion is produced by guanine oxidation is 7,8-dihydro-8-oxoguanine (8-oxoG). Oxidizing agents can also react with sugar-phosphate backbone of the DNA, leading to chemical modifications to the sugar moiety of the backbone, e.g., deoxyribose, which can cause eventual formation of single-strand breaks or double-strand breaks. Oxidative DNA damage, when not repaired by the cellular DNA repair machinery, can lead to mutations, cellular growth arrest, or cell death.

The ability of cancer cells to repair oxidative DNA damage is dependent on cellular mechanisms that include Base Excision Repair (BER), Nucleotide Excision Repair (NER), Mismatch Repair (MMR), homologous recombination or non-homologous end joining. The cancer characterized by an impaired ability to repair oxidative DNA damage may have a phenotype which is characteristic of a deficiency in one or more components of BER, NER, MMR, homologous recombination or non-homologous end joining, i.e. activity of one or more components of BER, NER, MMR, homologous recombination or non-homologous end joining is reduced or abolished in the cancer. Cancer with such a phenotype may be deficient in one or more components of the BER, NER, MMR, homologous recombination or non-homologous end joining, i.e., the expression and/or activity of the component may be reduced or abolished in the cancer, for example by means of mutation, polymorphism or epigenetic modification, such as hypermethylation, in the encoding nucleic acid or in a gene encoding a regulatory factor. In some embodiments, the impaired ability to repair oxidative DNA damage comprises impaired base excision repair (BER). In alternative embodiments, the impaired ability of cancer to repair oxidative DNA damage comprises impaired Nucleotide Excision Repair (NER), Mismatch Repair (MMR), homologous recombination or non-homologous end joining.

BER can act to remove small non-distorting base lesions, such as 8-oxoguanine, from DNA. The first step in BER can comprise hydrolysis of the glycosydic bond linking the damaged DNA base to the sugar in the DNA backbone, thereby resulting in removal of the damaged base from DNA. The first step can occur spontaneously or can be accomplished by a DNA glycosylase. Non-limiting examples of DNA glycosylases that can act on oxidatively damaged DNA in humans include 8-oxoguanine DNA glycosidase (hOGG1) and A/G-specific DNA glycosylase (hMYH). After the base lesion is removed, an abasic site is created in the DNA helix. The second step of the BER process involves hydrolysis of the phosphodiester bond located 5′ to the abasic site, thereby resulting in creation of a single-strand break. The second step can be accomplished by the AP lyase activity of DNA glycosylase or by AP endonuclease. After the oxidatively damaged base is converted into a single-strand break, it can be repaired by either long-patch repair or short patch repair mechanisms. The long-patch repair mechanism involves the activities of BER enzymes DNA polymerase for displacing synthesis, Flap endonuclease and DNA ligase. The short-patch repair mechanism involves the activities of BER enzymes 5′-dRP lyase (dRpase, DNA polymerase β), DNA polymerase for non-displacing synthesis and of a DNA ligase. Poly (ADP-ribose) polymerase (PARP) is also involved in BER and functions by recognizing single-strand breaks created in the second step of BER as described above, and by recruiting other repair enzymes, e.g., dRpase, to the site of the single-strand break, thereby promoting DNA repair.

An impairment in BER can arise when the activity of one or more components involved in BER, e.g., glycosylase (such as hOGG1 or hMYH), AP endonuclease, Flap endonuclease, DNA ligase, 5′-dRP lyase, or PARP is reduced or abolished in cancer. Cancer, for example, may be deficient in glycosylase, such as (hOGG1 or hMYH), AP endonuclease, Flap endonuclease, DNA ligase, 5′-dRP lyase, and/or PARP, i.e., expression and/or activity of glycosylase (such as hOGG1 or hMYH), AP endonuclease, Flap endonuclease, DNA ligase, 5′-dRP lyase, or PARP may be reduced or abolished in the cancer cells, for example by means of mutation, polymorphism or epigenetic modification, such as hypermethylation, in the encoding nucleic acid or in a gene encoding a regulatory factor. In one embodiment, expression and/or activity of hOOG1 glycosylase is impaired in a cancer that is characterized by an impaired ability to repair oxidative DNA damage.

In some embodiments, the cancer characterized by an impaired ability to repair oxidative DNA damage is breast cancer. Breast cancer is composed of five major subtypes, with subtype classification based on microarray gene classifications, as described in Perou et al., Molecular portraits of human breast tumors, Nature 2000, 406, 747-52 and in Sorlie et al., Gene expression patterns of breast carcinomas distinguish tumor subclasses with clinical implications, Proc. Natl. Acad. Sci. USA 2001, 98:10869-74, the entire contents of which are incorporated herein by reference. The breast cancer subtypes can be luminal A, luminal B, normal breast-like, human epidermal growth factor receptor 2 (HER2), and basal-like breast cancers. Basal-like breast cancer can be particularly sensitive to oxidative DNA damage, and this sensitivity can be caused by the impaired ability of basal-like breast cancer to repair oxidative DNA damage by BER, as discussed above. Basal-like breast cancer has been previously identified as a cancer characterized by impaired BER, as described in Alli et al., Defective repair of oxidative DNA damage in triple-negative breast cancer confers sensitivity to inhibition of Poly(ADP-ribose) Polymerase, Cancer Res., 2009, 69, 3589-96, the entire contents of which are incorporated herein by reference. Accordingly, in some embodiments, the present invention provides methods of treating cancer with bis(thiohydrazide amide) compounds, wherein the cancer characterized by the impaired ability to repair oxidative DNA damage is basal-like breast cancer.

The basal-like subtype of breast cancer can encompass BRCA1-deficient breast cancers. BRCA1 is a known tumor suppressor and its association with breast cancer is well known to one of skill in the art. BRCA1-deficient cancers may have BRCA1 deficient phenotype, i.e. BRCA1 activity is reduced or abolished in the cancer cells. Cancer cells with this phenotype may be deficient in BRCA1, i.e. expression and/or activity of BRCA1 may be reduced or abolished in the cancer cells, for example, by means of mutation, polymorphism or epigenetic modification, such as hypermethylation, in the encoding nucleic acid or in a gene encoding a regulatory factor. BRCA1-deficient breast cancer has been previously identified as a cancer characterized by impaired BER, as described in Alli et al., Defective repair of oxidative DNA damage in triple-negative breast cancer confers sensitivity to inhibition of Poly(ADP-ribose) Polymerase, Cancer Res., 2009, 69, 3589-96. Accordingly, in some embodiments, the present invention provides methods of treating cancer with bis(thiohydrazide amide) compounds, wherein the cancer characterized by an impaired ability to repair oxidative DNA damage is BRCA 1-mutated.

Basal-like sporadic breast cancers and BRCA1-mutated hereditary breast cancers can assume triple-negative status, e.g., these cancers can lack expression of estrogen receptor α (ER-α), and progesterone receptor (PR) and can also lack the amplification/overexpression of the HER2/Neu oncogene. Triple-negative breast cancer has been previously identified as a cancer characterized by impaired BER, as described in Alli et al., Defective repair of oxidative DNA damage in triple-negative breast cancer confers sensitivity to inhibition of Poly(ADP-ribose) Polymerase, Cancer Res., 2009, 69, 3589-96. Accordingly, in another embodiment, the present invention provides methods of treating cancer with bis(thiohydrazide amide) compounds, wherein the cancer characterized by an impaired ability to repair oxidative DNA damage is triple-negative breast cancer.

A cancer may be identified as having an impaired ability to repair oxidative DNA damage, for example, by determining the activity of the oxidative DNA damage repair pathways, e.g., BER, NER, MMR, homologous recombination and/or non-homologous end joining, in one or more cancer cells from a sample obtained from the individual, or by determining the activity of one or more components of the oxidative DNA damage repair pathways, e.g., BER, NER, MMR homologous recombination and/or non-homologous end joining. For example, a cancer may be identified as having impaired ability to repair oxidative DNA damage by BER by determining the activity of BER-specific components, e.g., glycosylase (such as hOGG1 or hMYH), AP endonuclease, Flap endonuclease, DNA ligase, 5′-dRP lyase, or PARP. Activity may be determined relative to normal (i.e. non-cancer) cells, preferably from the same tissue.

The activity of the oxidative DNA damage repair pathways, e.g., BER, may be determined, e.g., by a Green Fluorescent Protein (GFP)-based assay BER assay as described in Alli et al., Defective repair of oxidative DNA damage in triple-negative breast cancer confers sensitivity to inhibition of Poly(ADP-ribose) Polymerase, Cancer Res., 2009, 69, 3589-96. The GFP-based BER assay consists of the basic steps of a) oxidatively damaging a GFP-reporter gene, b) adenoviral-mediated gene transfer for delivery of the damaged GFP-reporter gene into living cells, and 3) host-cell reactivation, which allows for repair of the oxidatively-damaged reporter gene and expression of GFP and 4) measuring the fluorescent signal from GFP. Other methods for determining the activity of the oxidative DNA damage repair pathways, e.g., BER, may include measurements of sensitivity of cancer to oxidizing agents, such as hydrogen peroxide, as well as the use of western blot analysis, immunohistology, chromosomal abnormalities, enzymatic or DNA binding assays and plasmid-based assays to monitor for the protein components of oxidative DNA damage repair pathways, e.g., BER. In addition, genome-forward approaches to personalized oncology that may include using next-generation DNA sequencing technologies to identify genomic characteristics of individual tumors may be used to discover mutations in DNA repair genes predictive of sensitivity to oxidative DNA damage (e.g. mutations in OGG1 or other BER genes).

A cancer may be identified as having an impaired ability to repair oxidative DNA damage, e.g., having impaired BER, by determining the presence in cancer cells from the individual of one or more variations, for example, polymorphisms or mutations, in a nucleic acid encoding a polypeptide which is a component of the oxidative DNA damage repair pathways, e.g., BER. Such components can include a glycosylase (such as hOGG1 or hMYH), AP endonuclease, Flap endonuclease, DNA ligase, 5′-dRP lyase, or PARP.

Sequence variations such as mutations and polymorphisms may include a deletion, insertion or substitution of one or more nucleotides, relative to the wild-type nucleotide sequence. The one or more variations may be in a coding or non-coding region of the nucleic acid sequence and, may reduce or abolish the expression or function of oxidative DNA damage repair proteins, e.g., BER components as listed above. In other words, the variant nucleic acid may encode a variant polypeptide which has reduced or abolished activity or may encode a wild-type polypeptide which has little or no expression within the cell, for example through the altered activity of a regulatory element. A variant nucleic acid may have one, two, three, four or more mutations or polymorphisms relative to the wild-type sequence.

The presence of one or more variations in a nucleic acid which encodes a protein that functions in oxidative DNA damage repair, e.g., a BER component, may be determined by detecting, in one or more cells of a test sample, the presence of an encoding nucleic acid sequence which comprises the one or more mutations or polymorphisms, or by detecting the presence of the variant component polypeptide which is encoded by the nucleic acid sequence.

Various methods are available for determining the presence or absence in a sample obtained from an individual of a particular nucleic acid sequence, for example a nucleic acid sequence which has a mutation or polymorphism that reduces or abrogates the expression or activity of a protein that functions in oxidative DNA damage repair, e.g., a BER component. Furthermore, having sequenced nucleic acid of an individual or sample, the sequence information can be retained and subsequently searched without recourse to the original nucleic acid itself. Thus, for example, scanning a database of sequence information using sequence analysis software may identify a sequence alteration or mutation.

The methods for determining the presence or absence in a sample obtained from an individual of a particular nucleic acid sequence are well known to one of skill in the art and may comprise determining the binding of an oligonucleotide probe to nucleic acid obtained from the sample, for example, genomic DNA, RNA or cDNA. The probe may comprise a nucleotide sequence which binds specifically to a nucleic acid sequence which contains one or more mutations or polymorphisms and does not bind specifically to the nucleic acid sequence which does not contain the one or more mutations or polymorphisms, or vice versa.

The oligonucleotide probe may comprise a label and binding of the probe may be determined by detecting the presence of the label. A method may include hybridization of one or more (e.g. two) oligonucleotide probes or primers to target nucleic acid. Where the nucleic acid is double-stranded DNA, hybridization will generally be preceded by denaturation to produce single-stranded DNA. The hybridization may be as part of a PCR procedure, or as part of a probing procedure not involving PCR. An example procedure would be a combination of PCR and low stringency hybridization.

Binding of a probe to target nucleic acid (e.g. DNA) may be measured using any of a variety of techniques at the disposal of those skilled in the art. For instance, probes may be radioactively, fluorescently or enzymatically labeled. Other methods not employing labeling of probe include examination of restriction fragment length polymorphisms, amplification using PCR, RNase cleavage and allele specific oligonucleotide probing. Probing may employ the standard Southern blotting technique. For instance, DNA may be extracted from cells and digested with different restriction enzymes. Restriction fragments may then be separated by electrophoresis on an agarose gel, before denaturation and transfer to a nitrocellulose filter. Labeled probe may be hybridized to the DNA fragments on the filter and binding determined.

Nucleic acid, which may be genomic DNA, RNA or cDNA, or an amplified region thereof, may be sequenced to identify or determine the presence of polymorphism or mutation therein. A polymorphism or mutation may be identified by comparing the sequence obtained with the database sequence of the component, as set out above. In particular, the presence of one or more polymorphisms or mutations that cause abrogation or loss of function of the polypeptide component, and thus the oxidative DNA damage repair pathway as a whole, may be determined Sequencing may be performed using any one of a range of standard techniques well known to one of skill in the art.

A specific amplification reaction such as PCR using one or more pairs of primers may conveniently be employed to amplify the region of interest within the nucleic acid sequence, for example, the portion of the sequence suspected of containing mutations or polymorphisms. The amplified nucleic acid may then be sequenced as above, and/or tested in any other way to determine the presence or absence of a mutation or polymorphism which reduces or abrogates the expression or activity of one of proteins that function in oxidative DNA damage repair, e.g., BER components.

In some embodiments, a cancer may be identified as deficient in oxidative DNA damage repair by assessing the level of expression or activity of a positive or negative regulator of a protein that functions in oxidative DNA damage repair, e.g., a BER component. Expression levels may be determined, for example, by Western blot, ELISA, RT-PCR, nucleic acid hybridization or karyotypic analysis. In other embodiments, subcellular localization of BER enzymes or regulatory components may also be indicative that function in BER may also be indicative of BER deficiency. For example, absence of BER enzymes from the nucleus where they are normally localized may be indicative of BER deficiency.

Methods of identifying triple-negative breast cancers and BRCA-1 negative breast cancers are well-known in the art and may include immunohistochemistry (IHC), fluorescence in situ hybridization (FISH), chromogenic in situ hybridization (CISH), enzyme-linked immunosorbent assays (ELISA). Methods of identifying basal-like breast cancers may include gene expression profiling as described in Prat et al., Practical implications of gene-expression-based assays for breast oncologists, Nat. Rev. Clin. Oncol., 2012, 9, 48-57 and in Perou et al., Molecular stratification of triple-negative breast cancers, Oncologist 2011, 16 (Suppl. 1), 61-70.

In some embodiments, the present invention provides the methods of predicting sensitivity of cancer to treatment with bis(thiohydrazide amide) compounds based on its ability to repair oxidative DNA damage. An impaired ability to repair oxidative DNA damage is indicative of increased sensitivity of the cancer to treatment with bis(thiohydrazide amide) compounds. Assessment of the ability of cancer to repair oxidative DNA damage can be accomplished by any method as described above. In some embodiments, provided are methods of treating cancer in a subject in need thereof, comprising the steps of:

a) assessing sensitivity of the cancer to bis(thiohydrazide amides) compounds by assessing the ability of the cancer to repair oxidative DNA damage;

b) if the cancer is sensitive to bis(thiohydrazide amide) compounds, treating the subject with cancer by administering bis(thiohydrazide amide) compounds; and

c) if the cancer is not sensitive to bis(thiohydrazide amide) compounds, treating the subject with cancer by administering an anti-cancer therapy that does not comprise administering a bis(thiohydrazide amide) compound.

The term “subject” refers to human and non-human animals, including veterinary subjects. The term “non-human animal” includes all vertebrates, e.g., mammals and non-mammals, such as non-human primates, mice, rabbits, sheep, dog, cat, horse, cow, chickens, amphibians, and reptiles. In a preferred embodiment, the subject is a human and may be referred to as a patient.

“Treating a subject with a cancer” includes achieving, partially or substantially, one or more of the following: arresting the growth or spread of a cancer, reducing the extent of a cancer (e.g., reducing size of a tumor or reducing the number of affected sites), inhibiting the growth rate of a cancer, ameliorating or improving a clinical symptom or indicator associated with a cancer (such as tissue or serum components) and/or reducing the likelihood of the cancer recurring once it has been removed or gone into remission.

The term “effective amount” is the quantity of a bis(thiohydrazide amide) compound required to maintain desired concentration of a bis(thiohydrazide amide) compound in a subject while being effective for treating cancer that is characterized by an impaired ability to repair oxidative DNA damage. In some embodiments, the effective amount of bis(thiohydrazide amide) compound is sufficient to maintain the desired plasma or serum concentration of the bis(thiohydrazide amide) compound. The precise amount of the bis(thiohydrazide amide) compound to be administered to a subject will depend on the bis(thiohydrazide amide) compound levels that are to be achieved and/or maintained in a subject, as well as on the exact mode of administration, as discussed above. When bis(thiohydrazide amide) compound is co-administered with another anti-cancer agent, e.g., a taxane, for the treatment of cancer characterized by an impaired ability to repair oxidative DNA damage, an “effective amount” of the second anti-cancer agent will depend on the type of drug used. Suitable dosages are known for approved anti-cancer agents and can be adjusted by the skilled artisan according to the condition of the subject, the type of cancer being treated and the compound of the invention being used.

The terms “administer”, “administering” or “administration” include any method of delivery of a pharmaceutical composition or agent into a subject's system or to a particular region in or on a subject. In certain embodiments of the invention, an agent is administered intravenously, intramuscularly, subcutaneously, intradermally, intranasally, orally, transcutaneously, or mucosally. In a preferred embodiment, an agent is administered systemically. Administering an agent can be performed by a number of people working in concert. Administering an agent includes, for example, prescribing an agent to be administered to a subject and/or providing instructions, directly or through another, to take a specific agent, either by self-delivery, e.g., as by oral delivery, subcutaneous delivery, intravenous delivery through a central line, etc.; or for delivery by a trained professional, e.g., intravenous delivery, intramuscular delivery, intratumoral delivery, etc.

In some embodiments, a bis(thiohydrazide amide) compound is administered as a monotherapy, e.g., as the only anticancer drug administered to a subject to treat cancer. In a further embodiment, the bis(thiohydrazide amide) compound is continuously administered as a monotherapy. The term “continuously administered” refers to a mode of administration, wherein constant concentration of a bis(thiohydrazide compound) is achieved and/or maintained in a subject for the duration of administration. The term “constant drug concentration” means that a given measurement of the drug concentration in a subject is within 5%, 10%, 15% or 20% of the desired therapeutic concentration of the drug. In another embodiment, the constant concentration of a bis(thiohydrazide amide) compound is the concentration sufficient to achieve desired treatment objectives, e.g., achieving, partially or substantially, one or more of the following: arresting the growth or spread of a cancer, reducing the extent of a cancer (e.g., reducing size of a tumor or reducing the number of affected sites), inhibiting the growth rate of a cancer, ameliorating or improving a clinical symptom or indicator associated with a cancer (such as tissue or serum components) and/or reducing the likelihood of the cancer recurring once it has been removed or gone into remission. In one embodiment, the bis(thiohydrazide amide) compound continuously administered as a monotherapy is elesclomol represented by the following structural formula:

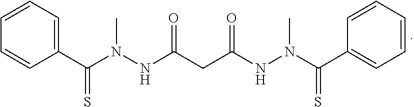 [SMILES: CN(NC(=O)CC(=O)NN(C)C(=S)c1ccccc1)C(=S)c1ccccc1]

Combination of Bis(Thiohydrazide Amide) Compounds with Other Agents for Treating Cancer

In some embodiments, the bis-thiohydrazide amide, when used for treating cancer characterized by an impaired ability to repair oxidative DNA damage, is administered in combination with an effective amount of another chemotherapeutic agent. In further embodiments, another chemotherapeutic agent can be a taxane, e.g., paclitaxel, cisplatin, gemcitabine or a PARP inhibitor.

Taxanes comprise a class of anti-cancer drugs that can act by enhancing and stabilizing microtubule formation. The term “taxane” is meant to include paclitaxel (or “Taxol™”) and paclitaxel analogs. “Paclitaxel analog” is defined herein to mean a compound which has the basic paclitaxel skeleton and which stabilizes microtubule formation. Many paclitaxel analogs are known, including docetaxel (Taxotere™). Paclitaxel and docetaxel have the respective structural formulas:

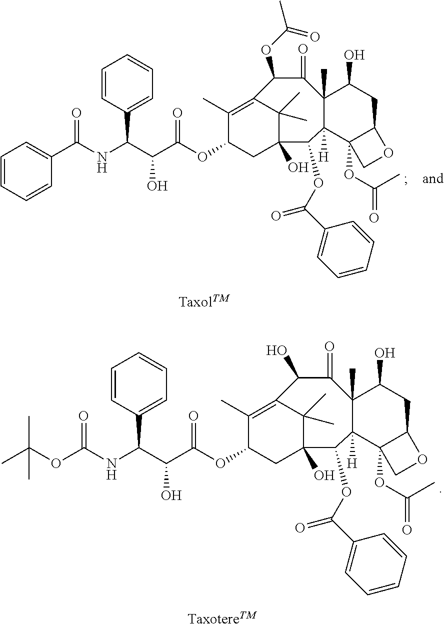

Various structural features of Taxol™ analogs have been described in previous publications, e.g., US 2009/0137682, the entire contents of which are incorporated herein by reference.

Platinum-containing compounds are currently widely used as chemotherapeutic agents and include cisplatin (Platinol or Platinol-AQ), carboplatin (Paraplatin or Paraplatin-AQ) and oxaliplatin (Eloxatin or Oxaliplatin Medac). Platinum-containing compounds act by damaging DNA, e.g., by forming DNA cross-links and eliciting cell cycle arrest and eventual apoptosis. Cisplatin, the first member in this class, is also referred to as cisplatinum or cis-diamminedichloroplatinum (II) is a compound represented by the following structural formula:

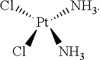

Nucleoside analogs represent an important class of chemotherapeutic agents. Gemcitabine (Gemzar) is a nucleoside analog and a chemotherapeutic agent that, when incorporated into DNA, inhibits further DNA synthesis. Gemcitabine is represented by the following structural formula:

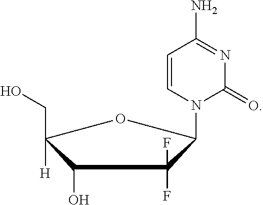

PARP inhibitors are agents and reduce or abolish the activity of poly(ADP-ribose) polymerase (PARP). Non-limiting examples of compounds that are known as PARP inhibitors include benzamides, e.g., 4-iodo-3-nitrobenzamide (Iniparib); benzimidazoles and indoles, e.g., 8-Fluoro-2-{4-[(methylamino)methyl]phenyl}-1,3,4,5-tetrahydro-6H-azepino[5,4,3-cd]indol-6-one (Rucaparib, AG-014699) and 2-((R)-2-Methylpyrrolidin-2-yl)-1H-benzimidazole-4-carboxamide (Veliparib, ABT-888); and phthalazin-1(2H)-ones and quinazolinones, e.g., 4-[(3-[(4-cyclopropylcarbonyl)piperazin-4-yl]carbonyl)-4-fluorophenyl]methyl(2H)phthalazin-1-one (Olaparib, AZD-2281), with structures as shown below:

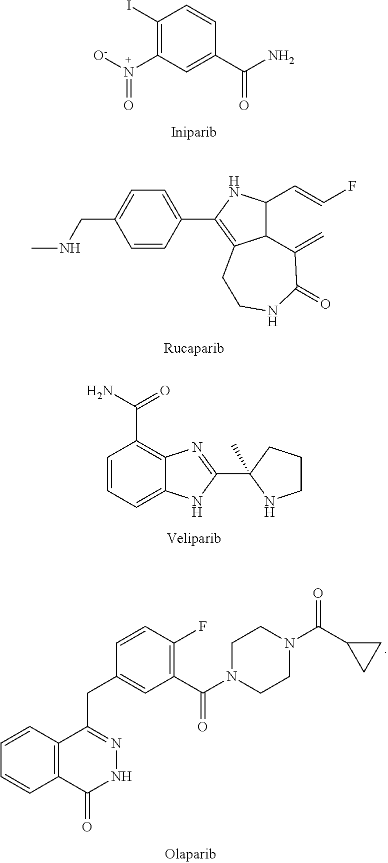

The dosages of chemotherapeutic agents to be administered with bis(thiohydrazide amide) compounds in accordance with the methods of the present invention will depend on the subject to be treated and the severity of the disease. The recommended dosages of chemotherapeutic agents can obtained from any reference in the art including, but not limited to, Hardman et al., eds., 1996, Goodman & Gilman's The Pharmacological Basis Of Basis Of Therapeutics 9^{th} Ed, Mc-Graw-Hill, New York; Physician's Desk Reference (PDR) 57^{th} Ed., 2003, Medical Economics Co., Inc., Montvale, N.J.

When a bis(thiohydrazide amide) compound is administered in combination with another chemotherapeutic agent, the therapies can be administered less than 5 minutes apart, less than 30 minutes apart, 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours apart.

The present invention is illustrated by the following examples, which are not intended to be limiting in any way.

### EXAMPLES

Elesclomol is a first-in-class investigational drug that exerts potent anticancer activity through the elevation of reactive oxygen species (ROS) levels and is currently under clinical evaluation as a novel anticancer therapeutic. Basal-like breast cancers, including those that contain germline mutations in BRCA1 gene, harbor compromised ability for repairing oxidative DNA damage. Defects in the ability of cancer cells to repair oxidative DNA damage by base excision repair pathway are predictive of the cells' sensitivity to elesclomol treatment.

### Materials and Methods

a. Cell Lines

Human breast cell lines represented the normal breast (MCF12A), basal-like breast cancer (BT549, HCC1806, MDAMB468), BRCA1-mutated breast cancer (HCC1937, SUM149PT, SUM1315MO2), and luminal breast cancer (BT474, MDAMB361, and T47D). Stable cell lines expressing shRNA to hOGG1 (shOGG1) or a non-targeting control (shCTRL) and BRCA1^{+/+} and BRCA1^{-/-} mouse mammary epithelial cells have been previously described (E. Alli, V. B. Sharma, P. Sunderesakumar, J. M. Ford, Defective repair of oxidative dna damage in triple-negative breast cancer confers sensitivity to inhibition of poly(ADP-ribose) polymerase, Cancer Res, 69, 2009, 3589-3596). All cell lines were maintained at 37° C. and 5% CO₂.

b. Measurement of Drug Sensitivity

Cells were exposed to increasing concentrations of elesclomol and incubated at 37° C. in the presence of 5% CO₂ for 96 hours. Cellular sensitivity was then determined by MTT assay and expressed as a percentage of the vehicle control (DMSO). Each data point represents the average of quadruplicate determinations +/−S.D. Images were obtained under 10× objective and are representative.

### Example 1

### Basal-Like and BRCA1-Mutated Breast Cancers Exhibit Greater Sensitivity to Elesclomol than the Luminal Breast Cancer or Normal Breast Cells

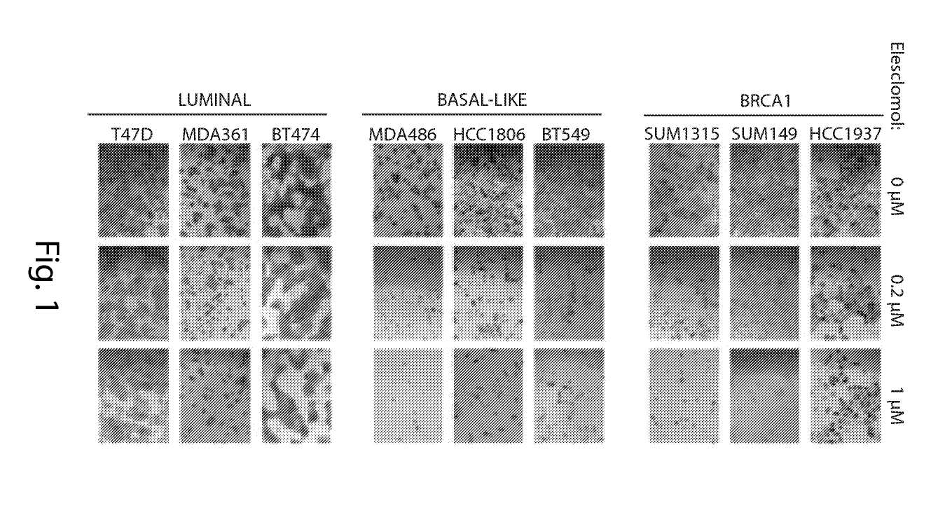

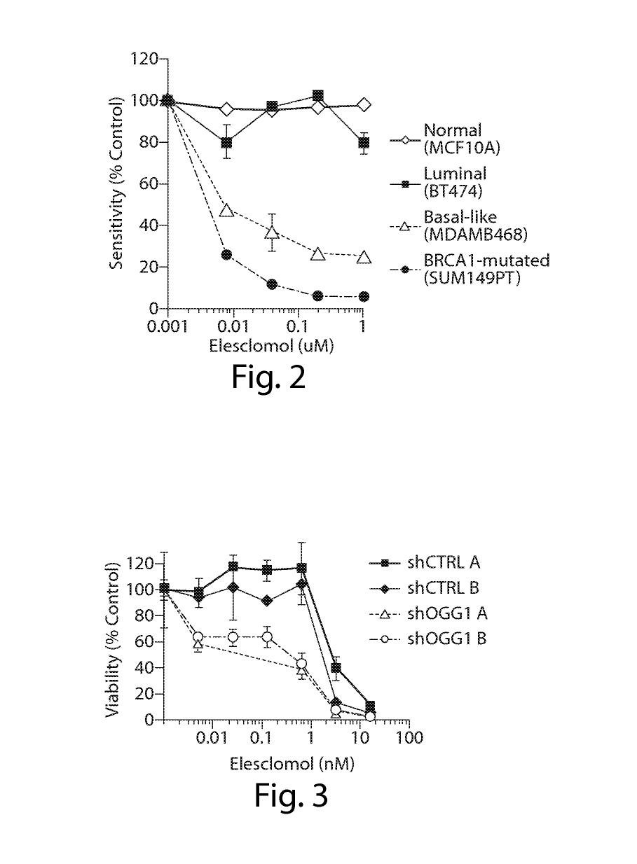

Cell lines that represent the normal breast or BRCA1-mutated, basal-like or luminal breast cancers were examined for sensitivity to elesclomol. Shown in FIG. 1 are images of BRCA-1 mutated, basal-like or luminal breast cancer cell cultures after treatment with 0 μM, 0.2 μM or 1 μM elesclomol. Shown in FIG. 2 is a cellular viability dose-response curve for normal or cancerous breast cells treated with increasing concentrations of elesclomol. This data demonstrates that basal-like and BRCA1-mutated breast cancers exhibit increased sensitivity to elesclomol, as compared to the normal breast cells or luminal breast cancer cells.

### Example 2

### BER-Compromised Cells are Characterized by the Increased Sensitivity to Elesclomol

Two different cell lines deficient in the hOOG1 DNA glycosylase that initiates base excision repair (shOGG1-A and sh-OGG1-B) and their isogenic control cells lines (shCTRL-A and shCTRL-B) were examined for sensitivity to elesclomol. FIG. 3 shows the resulting cell viability as a function of increasing elesclomol concentration, as measured by the MTT assay. The results demonstrate that compromised base excision repair correlates that the increased sensitivity to elesclomol.

What is claimed is:

1. A method of treating a subject with cancer characterized by an impaired ability to repair oxidative DNA damage, comprising administering to the subject an effective amount of a bis(thiohydrazide amide) compound, or a pharmaceutically acceptable salt thereof, or a deprotonated form thereof complexed to a transition metal cation, wherein:
the bis(thiohydrazide amide) compound is represented by the following Structural Formula: 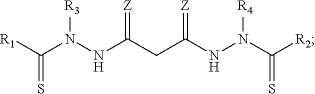
wherein:
R₁ and R₂ are each, independently, a phenyl optionally substituted with one or more groups selected from alkyl, haloalkyl, alkoxy, haloalkoxy, carbonyloxy, halogen, cyano, and nitro;
R₃ and R₄ are each, independently, —H or an alkyl;
each Z is independently O or S.

2. The method of claim 1, wherein the bis(thiohydrazide amide) compound is a disalt comprising two monovalent cations M⁺ or one divalent cation M²⁺.

3. The method of claim 2, wherein the bis(thiohydrazide amide) compound is a disalt comprising two monovalent cations M⁺.

4. The method of claim 3, wherein M⁺ is K⁺ or Na⁺.

5. The method of claim 1, wherein the bis(thiohydrazide amide) compound is a deprotonated form of the bis(thiohydrazide amide) compound complexed to a transition metal cation.

6. The method of claim 5, wherein the transition metal cation is Ni²⁺, Cu²⁺, Co²⁺, Fe²⁺, Zn²⁺, Pt²⁺ or Pd²⁺.

7. The method of claim 6, wherein the transition metal cation is Cu²⁺.

8. The method of claims 1-7, wherein R₃ and R₄ are both methyl and each Z is O.

9. The method of claims 1-8, wherein R₁ and R₂ are each phenyl optionally substituted with one or more groups consisting of methyl, ethyl, methoxy, ethoxy, halogen, or hydroxy.

10. The method of claims 1-9, wherein R₁ and R₂ are each unsubstituted phenyl.

11. A method of treating a subject with cancer characterized by impaired ability to repair oxidative DNA damage, comprising administering to the subject an effective amount of a bis(thiohydrazide amide) compound, or a pharmaceutically acceptable salt thereof, or a deprotonated form thereof complexed to a transition metal cation, wherein:
the bis(thiohydrazide amide compound) is represented by the following Structural Formula:

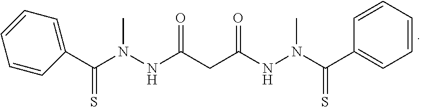 [SMILES: CN(NC(=O)CC(=O)NN(C)C(=S)c1ccccc1)C(=S)c1ccccc1]

12. The method of claim 1, wherein the bis(thiohydrazide amide) compound is a disalt comprising two monovalent cations M⁺ or one divalent cation M²⁺.

13. The method of claim 12, wherein the bis(thiohydrazide amide) compound is a disalt comprising two monovalent cations M⁺.

14. The method of claim 13, wherein M⁺ is K⁺ or Na⁺.

15. The method of claim 11, wherein the bis(thiohydrazide amide) compound is a deprotonated form of the bis(thiohydrazide amide) compound complexed to a transition metal cation.

16. The method of claim 15, wherein the transition metal cation is Ni²⁺, Cu²⁺, Co²⁺, Fe²⁺, Zn²⁺, Pt²⁺ or Pd²⁺.

17. The method of claim 16, wherein the transition metal cation is Cu²⁺.

18. The method of claims 1-17, wherein the cancer is breast cancer.

19. The method of claim 18, wherein the breast cancer is BRCA1-mutated breast cancer.

20. The method of claim 18, wherein the breast cancer is basal-like breast cancer.

21. The method of claim 18, wherein the breast cancer is triple-negative breast cancer.

22. The method of claim 1, wherein the impaired ability to repair oxidative DNA damage comprises impaired base excision repair (BER).

23. The method of any of claims 1-22, wherein the bis(thiohydrazide amide) compound is administered in combination with another chemotherapeutic agent.

24. The method of claim 23, wherein the agent is selected from the group consisting of cisplatin, gemcitabine, paclitaxel, or a PARP inhibitor.

25. The method of claims 1-22, wherein the bis(thiohydrazide amide) compound is administered as a monotherapy.

26. The method of claim 25, wherein the bis(thiohydrazide amide) compound is administered continuously.

27. A method of treating cancer in a subject in need thereof, comprising the steps of:
a) assessing the ability of the cancer to repair oxidative DNA damage;
b) if the cancer is sensitive to bis(thiohydrazide amide) compounds, treating the subject with cancer by administering bis(thiohydrazide amide) compounds according to claims 1-17 and 23-26; and
b) if the cancer is not sensitive to bis(thiohydrazide amide) compounds, treating the subject with cancer by administering an anti-cancer therapy that does not comprise administering bis(thiohydrazide amide) compounds.

28. The method of claim 27, wherein the cancer is breast cancer.

29. The method of claim 28, wherein the breast cancer is BRCA1-mutated hereditary breast cancer.

30. The method of claim 28, wherein the breast cancer is basal-like sporadic breast cancer.

31. The method of claim 28, wherein the breast cancer is triple-negative breast cancer.
